(19) United States
(12) Patent Application Publication
(10) Pub. No.: US 2006/0194723 A1
(43) Pub. Date: Aug. 31, 2006

(54) Novel medication treatment and delivery strategies for Alzheimer's Disease, other disorders with memory impairment, and possible treatment strategies for memory improvement

(76) Inventor: Michael David Rabinoff, San Jose, CA (US)

Correspondence Address: JEFFREY FURR, 253 N. MAIN STREET, JOHNSTOWN, OH, 43031, US

(21) Appl. No.: 10/906,636

(22) Filed: Feb. 28, 2005

(51) Int. Cl. A61K 38/18; A61K 31/7072; A61K 31/198; A61K 31/66

(52) U.S. Cl. 514/12; 514/114; 514/49; 514/561

(57) ABSTRACT

The present invention is a novel method for medication treatment for Alzheimer's Disease, Mild Cognitive Impairment, Age Associated Memory Impairment, other disorders with memory impairment, and for possible treatment strategies for memory improvement.

### BACKGROUND OF THE INVENTION

This invention relates to a method for medication treatment for Alzheimer's disease, Mild Cognitive Impairment, Age Associated Memory Impairment, other disorders with memory impairment, and for possible treatment strategies for memory improvement.

### BACKGROUND

Effective cures for Alzheimer's disease and other disorders with memory impairment have not been found. Current treatments, such as acetylcholinesterase inhibitors such as donepezil, galantamine and rivastigmine, or other agents such as memantine, while useful, have only slowed the progression of the illness, delaying but not stopping the progressive decline of functioning associated with Alzheimer's disease. Likewise, new treatments are needed for Mild Cognitive Impairment, Age Associated Memory Impairment, other disorders with memory impairment, and for possible treatment strategies for memory improvement.

Pharmacogenomic effects may impinge on effective treatment of the disorders. Pharmacogenomic effects may preclude the effectiveness of the actual use of a medication, medications or medication regimen. For example, pharmacokinetic genomic effects (including, but not limited to, genes that affect absorption, distribution, metabolism and excretion of the drug) may significantly affect treatment response. If an individual has genes that greatly speed specific drug metabolism, then at standard doses, the concentration may never get high enough to produce treatment response. Conversely, if the individual has genes that lead to very slow drug metabolism, then the concentration in the blood may be too high when using standard doses, and side effects, adverse reactions, and toxicity could develop. Likewise, pharmacodynamic genomic effects could lead to poor activity of the medication at desired sites of action in the body, so that there is poor treatment response. Conversely, pharmacodynamic genomic effects might lead to increased and too high activity at desired sites of action, producing side effects, adverse effects and toxicity. Prior experimental results from pharmacogenomics studies in the public literature have been conflicting. That may be because they don't take into account 1). brain activity, 2). multi gene—effects of combination of genes that affect pharmacokinetics and pharmacodynamics, etc. 3). nutritional and environmental factors that affect brain functioning, 4). current body biochemistry. and 5). other psychological and social factors, etc. To generate the very best treatment response, it may be required to use a comprehensive model for personalized treatment selection (i.e. the best models might include more comprehensive biopsychosocial factors and models). While analysis of every one of these factors are not needed to improve treatment response beyond those produced by current treatment standards, their incorporation in treatment selection may yield improved results.

### PRIOR ART

U.S. Pat. No. 6,787,519 by Huang, et al. and issued on Sep. 7, 2004, is for methods of treating disorders related to apoE. It discloses methods inhibiting formation of neurofibrillary tangles; and methods for treating disorders relating to apolipoprotein E (apoE) in a subject. The methods generally involve reducing the level of a carboxyl-terminal truncated form of apoE in a neuronal cell of a subject. The invention further provides isolated cells comprising a nucleic acid molecule encoding a carboxyl-terminal truncated form of apoE; and methods of screening compounds using the cells.

U.S. Pat. No. 6,670,369 by Xilinas, et al. and issued on Dec. 30, 2003, is for the use of phanquinone for the treatment of Alzheimer's disease. It discloses the use of phanquinone for the manufacture of a pharmaceutical composition for the prevention or the treatment of Alzheimer's disease is disclosed. Also methods of treatment or prevention of Alzheimer's disease are disclosed.

U.S. Pat. No. 6,576,672 by Murphy and issued on Jun. 10, 2003, is for a Polyamine treatment of neurological disorders. It discloses 2,3,2 Tetramine (3,7-diazanonane-1,9-diamine)propounded for treatment of Parkingson's Disease and dementias characterized by mitochondrial damage in view of this compound's ability to completely neutralize the dopamine-depriving effect of MPTP in laboratory animals up to 12 hours post MPTP injection, and to retain partial protection at suboptimal tissue levels for up to 36 hours. The effect of injecting combinations of MPTP and/or reducing agents and/or xenobiotics and/or depigmenting agents on Dopamine, Norepinephrine, Serotonin and Epinephrine levels demonstrated that MPTP and MPP+ act as reducing agents that mobilize copper and calcium, and sequester iron, and that the vulnerability of dopamine to these types of neurotoxins and to xenobiotics and metals can be corrected by administration of 2,3,2 tetramine that appears to redistribute metals between diverse storage pools and free metals in cytosol and regulate receptor mediated events, among other antidotal effects analogous to those of some of the endogenous polyamines.

U.S. Pat. No. 6,569,848 and issued on May 27, 2003 and U.S. Pat. No. 6,268,358 and issued on Jul. 31, 2001, by Davis, et al. is for compounds for the treatment of Alzheimer's disease.

U.S. Pat. No. 6,524,616 by Notelivitz, et al. and issued on Feb. 25, 2003, is for compositions and methods for treating or preventing neurodegeneration and cognitive decline and dysfunction associated with Alzheimer's disease, aging, other dementia related disorders and estrogen deficiency related conditions. It discloses a composition for improving memory and concentration in mammals with disorders associated with memory impairment. The composition comprises a combination of at least one phytoestrogen and at least one acetylcholinesterase inhibitor such as Huperzine A, or any derivative, analog, metabolite or combination thereof. The composition may further include at least one mammalian estrogen. The method of the invention comprises the co-administration of at least one phytoestrogen and at least one acetylcholinesterase inhibitor in a therapeutically effective amount. Additionally, a mammalian estrogen may be co-administered.

U.S. Pat. No. 6,410,747 by Greig, et al. and issued on Jun. 25, 2002, is for highly selective butyrylcholinesterase inhibitors for the treatment and diagnosis of Alzheimer's disease and dementias. It discloses a method for preventing or treating cognitive impairments associated with aging or Alzheimer's disease which comprises treating a patient at risk for having the cognitive impairment with an effective amount of a highly selective butyrylcholinesterase inhibitor.

U.S. Pat. No. 6,132,724 by Blum and issued on Oct. 17, 2000, is for an allelic polygene diagnosis of reward deficiency syndrome and treatment. It discloses an enhancement of attentional processing is attained by administration of an endorphinase inhibitor or enkephalinase inhibitor and optionally, a dopamine precursor, or a serotonin precursor, a GABA precursor, or an endorphin or enkephalinase releaser, or certain herbal compounds including Rhodiola rosea extract (Pharmaline) and/or Huperzine. These components promote restoration of normal neurotransmitter function and the components combined enhance the release of dopamine at the nucleus accumbens and are non-addictive.

U.S. Pat. No. 6,001,852 by Gerolymatos and issued on Dec. 14, 1999, is for Clioquinol for the treatment of Alzheimer's disease. It discloses a new pharmaceutical composition is disclosed that comprises clioquinol, vitamin B.sub.1 2, and, optionally, pharmaceutical acceptable carriers and/or excipients. The use of the pharmaceutical composition removes or alleviates the side effects of clioquinol.

U.S. Pat. No. 5,965,571 by Hutchinson and issued on Oct. 12, 1999, is for Cholinesterase inhibitors for treatment of Parkinson's disease. It discloses a treatment for Parkinson's disease with an at least one cholinesterase inhibitor. The cholinesterase inhibitor has been found to alleviate both any symptoms of dementia as well as to reduce rigidity and improve motor function.

U.S. Pat. No. 5,716,941 by Rabinoff and issued on Feb. 10, 1998, is for the use of methyl donor compounds to treat neurological dysfunction associated with immune defects. It discloses a method for treating a mammal with a demyelinating disease associated with an immunological disorder comprising administering methylcobalamin or cyanocobalamin.

U.S. Pat. No. 5,663,448 by Collard, et al. and issued on Sep. 2, 1997, is for aromatic acetylcholinesterase inhibitors. It discloses compounds of general formula (I), stereosiomers and pharmaceutically acceptable salts thereof, wherein each of Z and Z′ are independently H or F; Q is (a), CH(OH), (b); X is H, Br, Cl, F or CF.sub.3; Y is H, Br, Cl, F, OH, OR.sub.5, OC(O)R.sub.4, N.sub.3, CN, NO.sub.2, SO.sub.3 H, CO.sub.2 R.sub.4, NH.sub.2, NHR.sub.9, NR.sub.9 R′.sub.9, C(R.sub.6)(R.sub.7)(V′R.sub.8) or C(O)R.sub.7, provided that when both Z and Z′ are F, then Y is H or F; V and V′ are each independently CH.sub.2 or 0; R.sub.1 is H or CH.sub.3; R.sub.2, R.sub.9 and R′.sub.9 are each independently (C.sub.1-6)alkyl, or R.sub.2 and V-R.sub.3 taken together with the carbon atom to which they are attached form a 3-6 membered ring; R.sub.3, R.sub.6, R.sub.7 and R.sub.8 are each independently H, (C.sub.1-6)alkyl, or (C.sub.3-6)cycloalkyl; R.sub.4 is H, (C.sub.1-10)alkyl, (C.sub.0-4)alkylene aryl or (C.sub.3-8)cycloalkyl; and R.sub.5 is (C.sub.1-10)alkyl, benzyl, phenethyl or (C.sub.3-6)cycloalkyl, possess anticholinesterase activity and may be used in the treatment of Degenrativa Dementias.

U.S. Pat. No. 5,508,271 by Rabinoff and issued on Apr. 16, 1996, is for the treatment of neurological dysfunction with methylcobalamin. It discloses a method for treating neurological dysfunctions associated with AIDS or ARC comprising the administration of methylcobalamin, alone or in combination with methionine. Substantial increases in the number of T4+ T-lymphocytes, T8+ T-lymphocytes, or the ratio of T4+ to T8+ T-lymphocytes is also observed with this treatment.

U.S. Pat. No. 5,409,948 by Greig, et al. and issued on Apr. 25, 1995, is for a method for treating cognitive disorders with phenserine. It discloses an improved method of cholinomimetic therapy for cognitive impairments associated with aging and Alzheimer's disease comprising treating a patient with an effective amount of phenserine or a pharmaceutically acceptable salt and derivatives.

U.S. Pat. No. 5,137,712 by Kask, et al. and issued on Aug. 11, 1992, is for the use of S-adenosyl-L-methionine (SAMe) to reverse and/or prevent supersensitivity, tolerance and extrapyramidal side effects induced by neuroleptic treatment. It discloses a method for reversing or preventing the onset of tolerance and the development of extrapyramidal side effects in humans due to prolonged treatment with neuroleptics, comprising including S-adenosyl-L-methionine (SAMe) in the treatment regime. By utilizing SAMe in combination with tolerance-inducing neurolepitcs to maintain a minimum dosage of the drug while retaining its efficacy, the potential for the development of neuroleptic-induced, extrapyramidal side effects is minimized.

U.S. Pat. No. 5,091,391 by Aizenman, et al. and issued on Feb. 25, 1992, is for a method of resisting neurodegenerative disorders. It discloses a method for resisting neurological damage caused by overstimulation of the MDA receptor of nerve cells by glutamate includes exposing the NMDA receptors to an oxidizing agent to thereby diminish overall activity of the receptors following activation by glutamate. The oxidizing agent preferably is a material selected from the group consisting of pyrroloquinoline quinone and topa hydantoin.

U.S. Pat. No. 4,977,172 by Johnson, et al. and issued on Dec. 11, 1990, is for a method of treating the symptoms of cognitive decline in an elderly patient employing (S)-3-ethyl-4-[(1-methyl-1H-imidazol-5-yl)-methyl]-2-oxazolidinone. It discloses a (S)-3-Ethyl-4-[(1-methyl-1H-imidazol-5-yl)-methyl]-2-oxazolidinone or a pharmaceutically acceptable salt thereof is useful in the treatment of the symptoms of cognitive decline in an elderly patient suffering therefrom including the treatment of Alzheimer's disease.

U.S. Pat. No. 4,950,658 by Becker, et al. and issued on Aug. 21, 1990, is for a method of medical treatment of Alzheimer's disease. It discloses a method for improving memory and accompanying symptoms in patients with Alzheimer's disease and related disorders of memory. The method includes the steps of dosing a patient with 2,2-dichlorovinyl dimethyl phosphate sometimes referred to as dichlorvos or DDVP, or a precursor thereof maintaining said dosage at a level and over a period of time sufficient to create a concentration of DDVP in the brain whereby memory improvement occurs. A satisfactory precursor for this method is 2,2,2-trichloro-1-hydroxyethyl dimethyl phosphate sometimes referred to as metrifonate and 1,2-dibromo, 2 dichlorvoethyl dimethyl phosphate sometimes referred to as naled.

U.S. Patent Application 20040138296 by Robertson, et al. and published on Jul. 15, 2004, is for an Amyloid immunization and Cox-2 inhibitors for the treatment of Alzheimer's disease. It discloses compositions and methods for the treatment or prevention of Alzheimer's disease. More particularly, the invention provides a combination therapy for the treatment or prevention of Alzheimer's disease, wherein the therapy comprises administering to a subject an amyloid beta vaccine in combination with a cyclooxygenase-2 selective inhibitor.

U.S. Patent Application 20040138117 by Hudson, et al. and published on Jul. 15, 2004, is for a Human choline acetyltransferase. It discloses a Human choline acetyltransferase polypeptide and DNA (RNA) encoding such polypeptide and a procedure for producing such polypeptide by recombinant techniques is disclosed. Also disclosed are methods for utilizing such polypeptide for the treatment of cognitive and neurological deficiencies or mental disturbances such as degenerative nervous system disorders, for example, Alzheimer's Disease, ALS and other cholinergic defects, and antagonists for treating Parkinson's Disease and other disorders relating to an over-expression of acetylcholine. Also disclosed are diagnostic methods for detecting a mutation in the human Choline Acetyltransferase nucleic acid sequence.

U.S. Patent Application 20030166721 by Carelli, et al. and published on Sep. 4, 2003, is for a Choline derivative for the treatment of Alzheimer's disease. It discloses Choline derivatives useful in the treatment of the diseases related with central cholinergic.

There is still room for improvement in the art.

### SUMMARY

This invention uses a combination treatment for Alzheimer's disease, Pick's disease, age associated memory impairment, mild cognitive impairment, treatment of memory impairment in other disorders with memory impairment, and as a possible treatment strategy for memory improvement, utilizing an acetylcholinesterase inhibitor and agents to increase acetylcholine production, thus maximizing availability of acetylcholine in neural tissues. Rivastigmine, Aricept or Reminyl, or other anticholinesterase agents could be used, along with glycerylphosphorylcholine (GPC) and/or 5 prime cytidine diphosphocholine (5CDPC) (both of these compounds have neurotropic properties, and are potential sources for the choline subunit of acetylcholine or may serve to increase the available concentration of choline to be used in acetylcholine production), plus a methyl donor. There is some experimental work to suggest that addition of a methyl donor increases acetylcholine presence beyond simple availability of choline (there appears to be upregulation of Choline acetyltransferase activity with increased presence of methyl donors, whether by methyl cobalamin, S-adenosylmethionine, or other agents). Phosphatidylserine (PS) appears to enhance the activity of the combination treatment.

### DETAILED DESCRIPTION

The following description is demonstrative in nature and is not intended to limit the scope of the invention or its application of uses.

The current invention pertains to a novel method for medication treatment for Alzheimer's disease, Mild Cognitive Impairment, Age Associated Memory Impairment, other disorders with memory impairment, and for possible treatment strategies for memory improvement.

This invention was based on prior Document Disclosure Number 535087 stamped received Jul. 22, 2003, Document disclosure Number 547732 mailed Feb. 22, 2004, stamped received Feb. 27, 2004, and Provisional patent application No. 60/443,007 filed Jan. 29, 2003 and Provisional patent application No 60/489,260 filing date Jul. 22, 2003. No priority date is claimed.

This invention uses a combination treatment for Alzheimer's disease, Pick's disease, age associated memory impairment, mild cognitive impairment, treatment of memory impairment in other disorders with memory impairment, and as a possible treatment strategy for memory improvement, utilizing an acetylcholinesterase inhibitor and agents to increase acetylcholine production, thus maximizing availability of acetylcholine in neural tissues. Rivastigmine, Aricept or Reminyl, or other anticholinesterase agents could be used, along with glycerylphosphorylcholine (GPC) and/or 5 prime cytidine diphosphocholine (5CDPC) (both of these compounds have neurotropic properties, and are potential sources for the choline subunit of acetylcholine or may serve to increase the available concentration of choline to be used in acetylcholine production), plus a methyl donor. There is some experimental work to suggest that addition of a methyl donor increases acetylcholine presence beyond simple availability of choline (there appears to be upregulation of Choline acetyltransferase activity with increased presence of methyl donors, whether by methyl cobalamin, S-adenosylmethionine, or other agents). Phosphatidylserine (PS) appears to enhance the activity of the combination treatment.

While acetylcholinesterase inhibitors decrease the degradation of acetylcholine, also facilitating the production of acetylcholine might be a therapeutic consideration. Prior attempts with choline supplementation have not produced great results (with patients taking 10 plus grams per day and developing a fishy odor).

Some studies suggest adding methylcobalamin (MCBL) to phosphatidylcholine (PC) lowers the dose of PC that is needed. However, those doses are still truly massive. Various theories have been put forth why MCBL would be helpful if choline is already present. (If choline isn't present, it is known that S-adenosylmethionine (SAMe) is used to provide 3 methyl groups to phosphatidylethanolamine to form PC, and it is thought that MCBL may facilitate production of S-adenosylmethionine (SAMe)). One theory is that the methylcobalamin non-specifically repairs neural damage to facilitate acetylcholine production. However, one publication has shown that methylcobalamin leads to increased choline acetyltransferase concentration (ChAT). This may be caused by increased MCBL, SAMe, or choline leading to upregulation of the ChAT gene).

The following examples are offered by way of illustration and not by way of limitation.

### EXPERIMENTAL

### Example 1

All patients were on the listed stable dose of an anticholinesterase inhibitor for at least 3 months before addition of MCBL and/or GPC and/or 5CDP and/or PS.

73 year old female

Aricept (10 mg po qd)+MCBL (10 mg po bid)+GPC (100 mg po tid)

6 month MMSE improvement 2 point (19→21)

78 year old female

Aricept (5 mg. po qd)+SAMe (400 mg po bid)+5CDPC (100 mg po tid)+

PS (100 mg po bid)

6 month MMSE improvement 2 point (21→23)

72 year old male

Rivastigmine (4.5 mg po bid)+MCBL (5 mg. po bid)+GPC (200 mg po tid)+PS (100 mg po tid)

6 month MMSE improvement 3 point (21→24)

74 year old male

Galantamine (8 mg. po bid)+MCBL (5 mg po bid)+GPC (100 mg po tid)+PS (100 mg po bid)

6 month MMSE improvement 2 point (18→20)

(doses titrated up to listed final levels)

Cobalamin levels baseline all in normal range, folate baseline values all in normal range

Improvement in cobalamin levels, increased for all subjects, but in normal range

Possible side effects. All subjects complained of transient GI complaints: increased transient flatulence (3 out of 4), nausea (2 out of 4), transient diarrhea (1 out of 4), all returning to prior level by end of first month of addition of MCBL, SAMe, GPC, 5CDPC and/or PS. Two subjects also complained of transient sensations of dry eyes.

All publications and patent applications cited in this specification are herein incorporated by reference as if each individual publication or patent application were specifically and individually indicated to be incorporated by reference.

As to a further discussion of the manner of usage and operation of the present invention, the same should be apparent from the above description. Accordingly, no further discussion relating to the manner of usage and operation will be provided.

Although the foregoing invention has been described in some detail by way of illustration and example for purposes of clarity of understanding, it will be readily apparent to those of ordinary skill in the art in light of the teachings of this invention that certain changes and modifications may be made, such as a variation of dose of compounds and form and route of administration: that doses may be altered, and the compound formulation and route of administration may be varied, and, optionally, pharmaceutical acceptable carriers and/or excipients may be utilized, thereto without departing from the spirit or scope of the appended claims.

1. A method for treating Alzheimer's disease, mild cognitive impairment, age associated memory impairment, memory impairment due to other disorders, and a treatment strategy for memory improvement, said method comprising: using an acetylcholinesterase inhibitor in combination with other agents for increased therapeutic effect.

2. A method according to claim 1, wherein said agents that increase production of acetylcholine are used alone or in combination with other therapeutic agents.

3. A method according to claim 1, wherein said agents that increase concentration of acetylcholine are used alone or in combination with other therapeutic agents.

4. A method according to claim 1, wherein said agents that are neurotropic factors and/or nerve growth factors are used alone or in combination with other therapeutic agents.

5. A method according to claim 1, wherein said agents that are methyldonor compounds are used alone or in combination with other therapeutic agents

6. A method according to claim 1, wherein said agents consist of one or more of a group that consist of vitamins, or amino acids.

7. A method according to claim 1, wherein said agents that are facilitate cell membrane formation are used alone or in combination with other therapeutic agents.

8. A method whereby such treatment methods, according to any one of the claim 1, whereby pharmacogenomic analysis is combined with one or more of a group of acetylcholinesterase inhibitor, an agent that leads to increased acetylcholine production, an agent that leads to increased acetylcholine concentration, an agent to facilitate cell membrane formation, a neurotropic agent, a nerve growth agent, a methyl donor, a form of vitamin(s), a form of amino acid(s).

9. (modified) A method whereby such treatment methods, according to any one of the claim 1, whereby proteomic analysis is combined with one or more of a group of an acetylcholinesterase inhibitor, an agent that leads to increased acetylcholine production, an agent that leads to increased acetylcholine concentration, an agent to facilitate cell membrane formation, a neurotropic agent, a nerve growth agent, a methyl donor, a form of vitamin(s), a form of amino acid(s).

10. A method whereby such treatment methods, according to any one of the claim 1, whereby biochemical analysis is combined with one or more of a group the consist of an acetylcholinesterase inhibitor, an agent that leads to increased acetylcholine production, an agent that leads to increased acetylcholine concentration, and agent to facilitate cell membrane formation, a neurotropic agent, a nerve growth agent, a methyl donor, a form of vitamin(s), a form of amino acid(s).

11. A method whereby such treatment methods, according to any one of the claim 1, whereby CNS imaging method is combined with one or more of a group the consist of said acetylcholinesterase inhibitor, an agent that leads to increased acetylcholine production, an agent that leads to increased acetylcholine concentration, an agent to facilitate cell membrane formation, a neurotropic agent, a nerve growth agent, a methyl donor, a form of vitamin(s), a form of amino acid(s).

12. A method whereby such treatment methods, according to any one of the claim 1, whereby a selection of neurological or neuropsychiatric testing is combined with one or more of a group the consist of acetylcholinesterase inhibitor, an agent that leads to increased acetylcholine production, agent that leads to increased acetylcholine concentration, an agent to facilitate cell membrane formation, a neurotropic agent, a nerve growth agent, a methyl donor, a form of vitamin(s), or a form of amino acid(s).

13. A method whereby such treatment methods, according to any one of the claim 1, whereby psychodiagnostic testing is combined with one or more of a group that consists of acetylcholinesterase inhibitor, an agent that leads to increased acetylcholine production, an agent that leads to increased acetylcholine concentration, an agent to facilitate cell membrane formation, a neurotropic agent, a nerve growth agent, a methyl donor, a form of vitamin(s), or a form of amino acid(s).

14. A method whereby such treatment methods, according to any one of the claim 1, which include one or more of a selection of pharmacogenomic analysis, proteomic analysis, biochemical analysis, CNS imaging, neurological testing, neuropsychiatric testing, psychodiagnostic testing combined with said acetylcholinesterase inhibitor, an agent that leads to increased acetylcholine production, an agent that leads to increased acetylcholine concentration, an agent that leads to facilitate cell membrane formation, a neurotropic agent, a nerve growth agent(s), a methyl donor, a form of vitamin(s), or a form of amino acid(s).

15. A method whereby such treatment methods, according to any claim 1, whereby said acetylcholinesterase inhibitor, consists of one or more of a group that consists of Rivastigmine, Aricept or Reminyl.

16. A method whereby such treatment methods, according to any claim 1, whereby said neurotropic factor or nerve growth factor consists of one or more of a group that consists of glycerylphosphorylcholine, five prime cytidine diphosphocholine (5CDPC), brain derived neurotropic factor, or nerve growth factor.

17. A method whereby such treatment methods, according to any claim 1, whereby said methyl donor consists of one or more of a group that consists of methyl cobablamin, other cobalamins, methionine, or S-adenosylmethionine.

18. A method whereby such treatment methods, according to any claim 1, whereby said form of vitamin consists of one or more of a group that consists of methyl cobablamin, or other cobalamins.

19. A method whereby such treatment methods, according to any claim 1, whereby said form of amino acid consists of one or more of a group that consists of methionine, or S-adenosylmethionine.

20. A method whereby such treatment methods, according to any claim 1, whereby said agent that increases acetylcholine production or concentration consists of one or more of the group that consists of glycerylphosphorylcholine, five prime cytidine diphosphocholine (5CDPC), methylcobalamin, or S-adenosylmethionine.

21. A method whereby such treatment methods, according to any claim 1, whereby said agent that increases cell membrane formation consists of one or more of a group that consists of phosphatidylserine, methylcobalamin, or S-adenosylmethionine.
